(19) United States
(12) Patent Application Publication
(10) Pub. No.: US 2021/0082574 A1
(43) Pub. Date: Mar. 18, 2021

(54) NOTIFICATION TOOL FOR NEED ASSESSMENT OF EMERGENCY SERVICES AND PATIENT WELL-BEING CONFIRMATION

(71) Applicant: Fresenius Medical Care Holdings, Inc., Waltham, MA (US)

(72) Inventors: Gabriel E. Alejo, San Antonio, TX (US); John J. Donnelly, Denver, CO (US); Dorman B. Mannon, II, Farmersville, TX (US)

(21) Appl. No.: 17/017,261

(22) Filed: Sep. 10, 2020

Related U.S. Application Data
(60) Provisional application No. 62/900,071, filed on Sep. 13, 2019.

(51) Int. Cl. G16H 40/20; G16H 40/67; G16H 20/40; G06Q 50/26; H04W 4/90

(52) U.S. Cl. CPC G16H 40/20; G16H 40/67; H04W 4/90; G06Q 50/265; G16H 20/40

(57) ABSTRACT

A notification tool is described to rapidly establish 2-way communication to a large audience in an effort to ascertain the need for emergency services (“HELP”) or confirmation of well-being (“SAFE”) during emergent events, such as a hurricane, flood, and other natural or man-made disasters. The benefits derived from the design and deployment of the notification tool are primarily based in the efficient use of time and resources during periods of catastrophe and peril. The notification tool can be used by personnel not directly impacted by the emergency and to contact all individual patients simultaneously and/or in a short time period, overcoming the constraint of a call tree where one more operators dial patients sequentially. Responses requiring intervention can be seen immediately in the directed mailbox by multiple individuals and escalated as appropriate.

### RELATED APPLICATIONS

This application claims priority to U.S. Provisional Application 62/900,071, filed Sep. 13, 2019, entitled “TEXT NOTIFICATION TOOL FOR NEED ASSESSMENT OF EMERGENCY SERVICES AND PATIENT WELL-BEING CONFIRMATION,” which is incorporated herein by reference in its entirety.

### TECHNICAL FIELD

The disclosure generally relates to healthcare systems, devices, and methods, and generally to communication with patients of a medical care provider during emergent or disaster events.

### BACKGROUND

Medical devices such as dialysis machines are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance. Both HD and PD machines may include displays with touch screens or other user interfaces that display information of a dialysis treatment and/or enable an operator or patient to interact with the machine.

Patients who undergo dialysis treatments, both at home and in clinics, often provide contact information, including telephone numbers, to the dialysis care provider to enable them to be contacted by the dialysis care provider. In the event of emergent events, including natural disasters, the dialysis care provider may contact the dialysis patients to assess their condition. Typically, in emergent or disaster events, large scale contact of dialysis patients is performed using a call tree where one or more operators dial patients sequentially. Call trees, however, may be resource intensive and inefficient.

Accordingly, it would be desirable to provide a system that addresses the above-noted concerns and other issues.

### SUMMARY

According to the system described herein, a notification system comprises a connected health system comprising a network system that enables secure transmission of information between a patient device and a remote entity; and a notification tool comprising an application having a broadcaster access interface and a recipient access interface. The notification tool is configured to allow a user at the remote entity to create an emergency message, send the emergency message via the connected health system to multiple recipients at the same time, and manage an interface between a messaging application and the messaging access platform. The emergency message requests a response from the recipient that is entered by the recipient via the recipient access interface.

According further to the system described herein, a medical system comprises a medical device that is configured to perform a medical treatment; a connected health system comprising a network system that enables secure transmission of information between the medical device and a remote entity, wherein the information includes health related information; and a notification tool comprising an application having a broadcaster access interface and a recipient access interface. The notification tool is configured to allow a user at the remote entity to create an emergency message, send the emergency message via the connected health system to multiple recipients at the same time, and manage an interface between a messaging application and the messaging access platform. The emergency message requests a response from the recipient that is entered by the recipient via the recipient access interface.

According further to one or more implementations of the system described herein, the emergency message may be a patient safety check request or a patient well-being confirmation request. The notification tool may include a web-based application and may further comprise an interface to a workflow application. The patient device may be a mobile device, and the recipient access interface may be installed on the mobile device, and the mobile device may be a smart phone or tablet device. The patient device may be a treatment device, such as a dialysis machine, having a display, and the recipient access interface may be installed on the treatment device. The display of the dialysis machine may display information related to a treatment performed by the dialysis machine and the emergency message. The notification tool may further comprise a response processing component that processes responses received in response to the emergency message and facilitates responsive action.

### BRIEF DESCRIPTION OF THE DRAWINGS

By way of example, embodiments of the disclosed methods and devices will now be described, with reference to the accompanying drawings, in which:

FIG. 1 shows a schematic illustration of a solution architecture of a notification tool that may be representative of some implementations of the system described herein.

FIGS. 2A and 2B show example notification tool screens on displays of the notification initiator/broadcaster and a recipient using the notification tool that may be representative of some implementations of the system described herein.

FIG. 3 is a schematic illustration showing a network messaging system, including use of a connected health system network, that may be representative of some implementations of the system described herein.

FIG. 4 is a schematic illustration showing an example of an operating environment that may be representative of some implementations of the system described herein.

FIG. 5 is a schematic illustration showing an example of an operating environment that may be representative of some implementations of the system described herein.

FIG. 6 is a schematic illustration of an exemplary embodiment of a dialysis machine and a controller that may be representative of some implementations of the system described herein.

### DETAILED DESCRIPTION

The present embodiments will now be described more fully hereinafter with reference to the accompanying drawings, in which several exemplary embodiments are shown. The subject matter of the present disclosure, however, may be embodied in many different forms and types of methods and devices for dialysis machines and other potential medical devices and should not be construed as limited to the embodiments set forth herein. Rather, these embodiments are provided so that this disclosure will be thorough and complete, and willfully convey the scope of the subject matter to those skilled in the art. In the drawings, like numbers refer to like elements throughout.

According to implementations of the system described herein, a notification tool is described to rapidly establish 2-way communication to a large audience, including via text, also known as short messaging service or SMS, in an effort to ascertain the need for emergency services (“HELP”) or confirmation of well-being (“SAFE”) during emergent events, such as a hurricane, flood, and other natural or man-made disasters. The benefits derived from the design and deployment of the notification tool are primarily based in the efficient use of time and resources during periods of catastrophe and peril. The notification tool can be used by personnel not directly impacted by the emergency and to contact all individual patients simultaneously and/or in a short time period, overcoming the constraint of a call tree where one or more operators dial patients sequentially. Responses requiring intervention can be seen immediately in the directed mailbox by multiple individuals and escalated as appropriate.

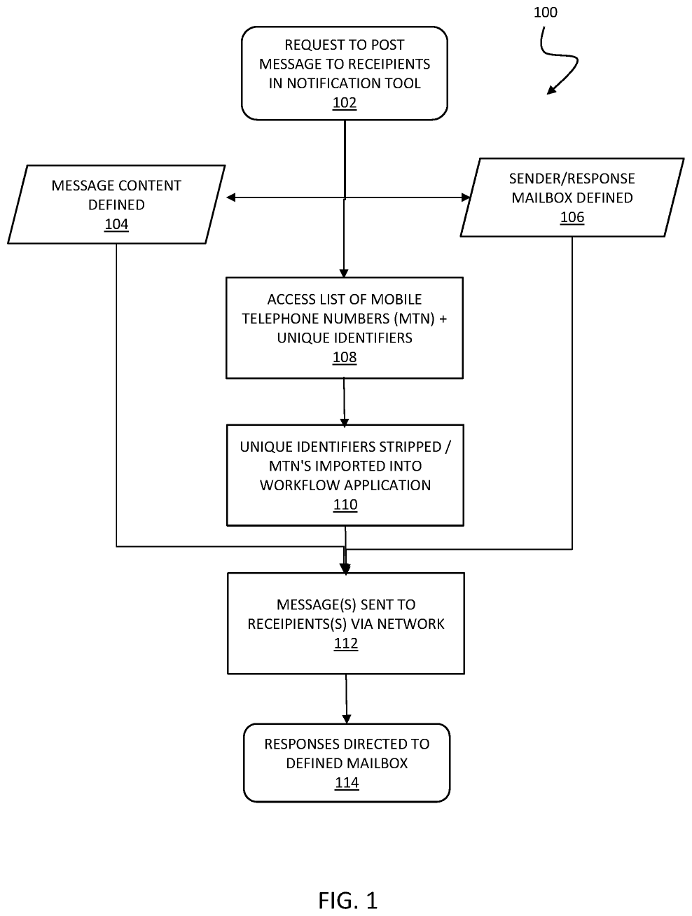

FIG. 1 shows, in an example implementation, a schematic illustration of the solution architecture 100 of the notification tool. The solution architecture 100 is based on the combination of three core technologies:
a workflow, such as a PerfectForms workflow;
a message access platform, such as a Verizon Enterprise Messaging Access Gateway (EMAG) subscription for access to SS7 Network; and
a messaging infrastructure, such as Microsoft Exchange SMTP Mail infrastructure and including one or more messaging or mail applications.

As shown, at a step 102, a request is initiated by the notification providing entity, e.g. healthcare providing entity, to post a safety check and/or well-being confirmation notification to recipients using the notification tool according to the system described herein. At a step 104, the message content is defined. At a step 106, the sender/response mailbox is defined. At a step 108, a list of mobile telephone numbers (MTNs) and unique identifiers associated with the users of the notification tool is accessed. At a step 110, the unique identifiers are stripped away, and the MTNs are imported into the workflow application, such as into a PerfectForms workflow application. At a step 112, a message is sent to recipients via a network, such as to SMTP Mail/SS7 network, using, for example, a message access gateway, such as a Verizon Enterprise Message Access Gateway (EMAG) subscription for access to SS7 Network, and a mail infrastructure, such as Microsoft Exchange SMTP Mail infrastructure that may include a messaging or mail application. Thereafter, at a step 114, response messages received from the notification tool users in response to the safety check/well-being confirmation are directed to a defined mailbox.

The notification tool may be implemented as Text Notification Tool (TNT) that provides a web-based application that enables enterprise customers to directly connect with carrier mobile device users by sending messaging content. The TNT Portal allows enterprise customers to: create and send emergency notifications; send a message to one, or hundreds, or even thousands of people at the same time; and manage the interface between the enterprise messaging applications and the EMAG platform.

In various implementations, it is noted that that the response text data received may be processed using natural language processing techniques. These techniques may include, but are not limited to word2vec, doc2vec, continuous bag of words, or others. This processed data may then be used to train a machine learning model to identify (e.g., predict) specific outcomes, e.g., coordinating with emergency provider personal to address any needed safety responses or well-being checks. It is further noted that the data communication and responses may be other than text, and include graphics, images and animations. It should be understood that some example embodiments may include other entities not shown, and/or may exclude some of the entities shown. Further, it should be understood that the illustrated communication channels are not exclusive, and the various entities may also, where appropriate, communicate directly or indirectly between each other and/or the patients. In some examples, the communication between the system and one or more of the other entities may be indirect, flowing through one or more intermediary entities.

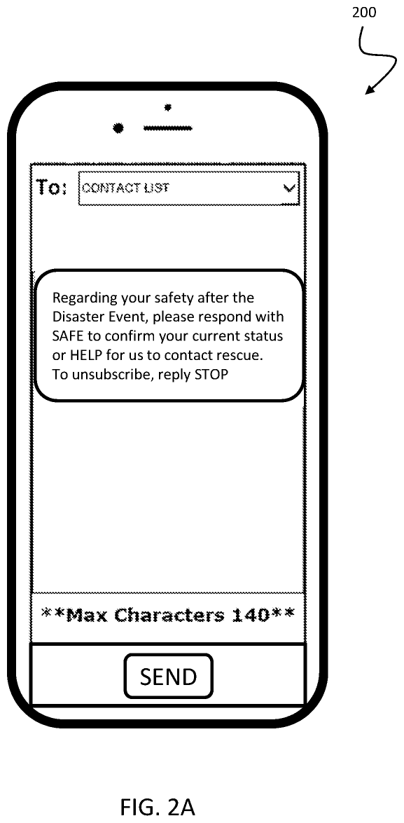

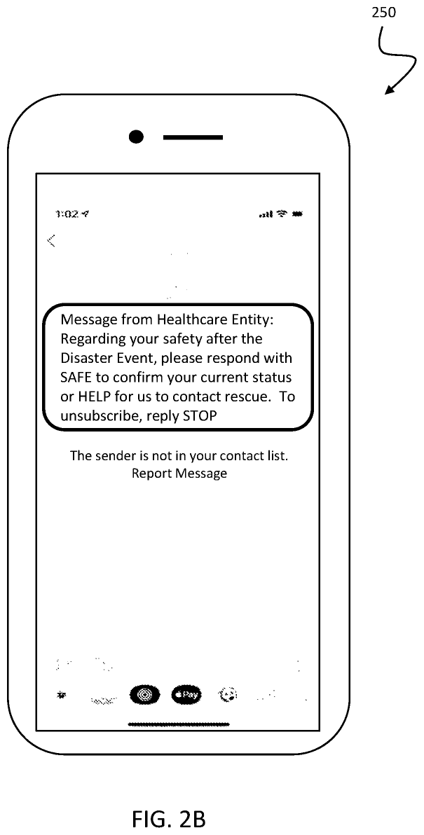

FIGS. 2A and 2B show example notification tool displays on screens of the notification initiator/broadcaster and a recipient using the notification tool according to an implementation of the system describe herein. FIG. 2A shows a broadcast notification tool screen on a display 200 of the notification broadcaster in connection with initiating the sending of a safety check and/or well-being confirmation message to users of the notification tool following a disaster event (e.g. hurricane, earthquake etc.). FIG. 2B shows a recipient notification tool screen on a display 250 of a notification recipient in connection with receiving the safety check and/or well-being confirmation message.

The system described herein may be used in connection with a notification broadcasting entity's (e.g. a healthcare provider entity) connected health system that provides remote network communication functions between users/patients (such as home dialysis patients) and hospital or clinic networks, and that may include connectivity transmissions between the notification broadcasting entity and notification tool via patient smartphones or tablet device and/or integrated into a home medical device, such as a home dialysis machine.

In an implementation, the notification tool may be a Text Notification Tool (TNT) that is a web-based application that enables enterprise customers to directly connect with carrier mobile device users by sending messaging content. The TNT Portal allows enterprise customers to: create and send emergency notifications; send a message to one, or hundreds, or even thousands of people at the same time; Manage the interface between the enterprise messaging applications and the EMAG platform TNT users send a contact list of Mobile Telephone Numbers (MTN)+Unique Identifiers, to the site administrator to load into the tool. A user can then send the desired message to the recipients which is processed by Verizon's EMAG (Enterprise Messaging Access Gateway) and converted to a form understandable by the Verizon Network elements. These messages are then sent to the Network elements for delivery to end devices as SMS (Standard Messaging System) messages regardless of the user's carrier. Responses back from the end devices as well as any delivery receipts received by EMAG are converted back into the protocol used by the enterprise customer's application and directed back to the customer defined email address.

In an implementation, a user of the notification broadcasting entity will need to be an approved user in the Access Control List (ACL) to be able to access the TNT application. When one message is sent to a group of contacts with FKC TNT Portal, the single message will be sent out/broadcasted by EMAG as individual messages to the predetermined contacts. Any replies coming from the contacts will be displayed as “one to one” message to the predefined email address. A notification broadcasting user may filter down to the group to which they would like to broadcast a message, as shown in FIG. 2A. The user may enter and view the message that they would like to send to this user list. For example, the message may read: “Regarding your safety after the Disaster Event, please respond with SAFE to confirm your current status or HELP for us to contact rescue. To unsubscribe, reply STOP.” Once the user has verified the message preview, the user may click the “Send” button. The message will be delivered to the targeted MTN and appear as shown in FIG. 2B. For example, the received message may read: “Message from Healthcare Entity: Regarding your safety after the Disaster Event, please respond with SAFE to confirm your current status or HELP for us to contact rescue. To unsubscribe, reply STOP.” In response, the recipient may then respond as appropriate and needed to the message, such as a message of “SAFE”. As further discussed elsewhere herein, responses may be reviewed and parsed by the notification broadcasting entity, including by automated processes, in connection with contacting, organizing and/or coordinating appropriate emergency responses.

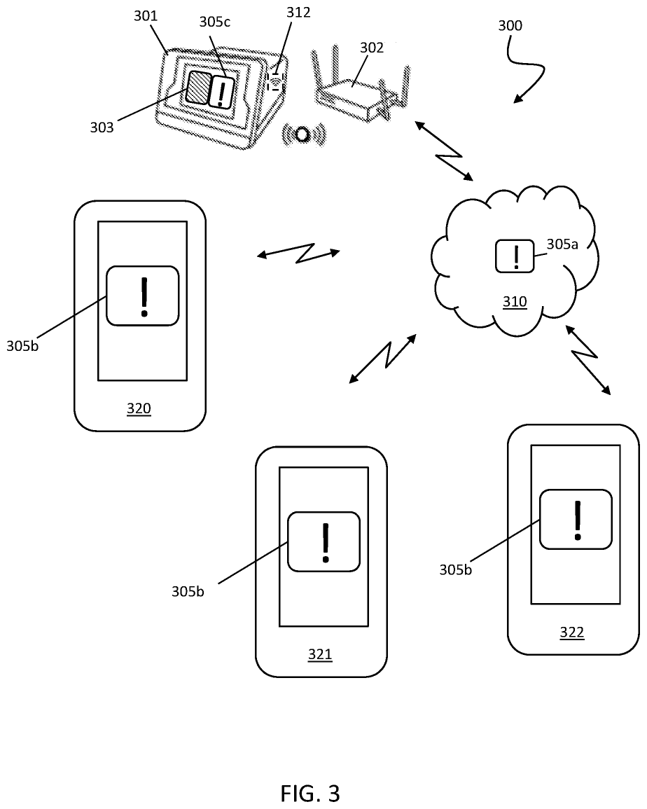

FIG. 3 is a schematic illustration showing a network messaging system 300, including use of a connected health network system, according to an implementation of the system described herein. A network 310 is illustrated schematically representing a network through which messages may be transmitted, such as a message 305a that may be an alert and/or other message concerning a need for emergency services or a request for well-being confirmation of a patient that may be transmitted from one or more entities and/or software systems to or via one or more components of a medical system including a dialysis machine 301. In various implementations, the network 310 may be a mobile telecommunications network and/or a cloud-based network that allows transmission of messages. The message is shown displayed as a message 305b on a mobile device 320 of the patient, and which may be the same as the message 305a or a different, expanded or condensed version of the message 305a. In an implementation, the message 305a may be short message service (SMS) or text message that is generated by or in connection with servers of a dialysis care provider and transmitted over the network messaging system 300 and sent in connection with an emergency or disaster situation. The message 305b may be the text message received on the mobile device 320 and which may invite a text-based response from the patient/recipient, such as “HELP” for a need for emergency services or “SAFE” indicating that the patient is in a safe situation.

The network messaging system 300, as described herein, may permit large scale notification to multiple patients/recipients, which is illustrated by multiple mobile devices 320, 321 and 322 in the figure. In various embodiments, the mobile devices 320, 321, 322 may include a smartphone, a tablet device and/or other mobile computing device having a display.

In an implementation, the message 305a may be transmitted via a cloud-based or wide area network communication system to the gateway device 302 and thereafter transmitted via a local area network, such as a Wi-Fi or Bluetooth connection, to the dialysis machine 301 and/or to the mobile device 320. Additionally, and/or alternatively, in another embodiment, the message 305a may be transmitted via a mobile telecommunication system to the mobile device 320 and thereafter transmitted via a local area network, such as a Wi-Fi or Bluetooth connection, to the dialysis machine 301 either directly or via the gateway 302. In an embodiment, the mobile device 320 may include and run a mobile application that enables communication between the mobile device 320 and the gateway 302 and/or dialysis machine 301. The communication may be suitably encrypted/decrypted and the processing may include authorized pairing between the mobile application/the mobile device 320 and the gateway 302 and/or the dialysis machine 301.

In another implementation, a version of the message 305a may also be received at and displayed on a dialysis machine 301, as message 305c, that may be connected to a network by one or more connection mechanisms of a connected health network system, including a connectivity device 312 of the dialysis machine 301 and/or by a gateway connectivity device 302 or other component of a connected health network system. The connected health network system may allow for the secure exchange of information, including health information, such as dialysis treatment information, between medical devices in the home of a patient and a remote entity. For further description of one or more example connected health system networks and operations thereof for securely transmitting medical information, reference is made to U.S. Pat. No. 10,623,188 B2 to Cohen et al., entitled “Securely Distributing Medical Prescriptions,” which is incorporated herein by reference in its entirety. The dialysis machine 301 may have a display that functions to enable control of a dialysis treatment, such as via a touch screen, and display of the dialysis treatment information. The emergency message 305c received at the dialysis machine, via the connected health system network and components, may enable the message to be received at the dialysis machine 301, and displayed along with the treatment information 303. In this way, a patient/recipient who is undergoing a treatment may be monitoring their treatment information 303 and may then be alerted to and view the emergency message 305c. The message 305c may invite a text-based response from the patient/recipient, such as “HELP” for a need for emergency services or “SAFE” indicating that the patient is in a safe situation that is input via an interface of the dialysis machine 310. Alternatively, the patient may unsubscribe by responding with “STOP.”

In various implementations, one or more of the messages 305a,b,c may include photographic, audio and/or video messages, and may include dynamic or interactive elements using graphical user interface (GUI) components (e.g. touchscreen) of the displaying screen, that, for example, require the patient to select options to proceed or respond through the interactive elements of the message, such as by responding by return text to a question posed in the message 505b.

According to implementations and embodiments of the system described herein, components of the network messaging system 300 may be used to send, transmit and/or otherwise convey the message 305a that may be an Internet message, a text or short message service (SMS) message, a multimedia messaging service (MMS) message, an email and/or other messaging path to inquire about the need for emergency services during an emergent or disaster situation and to request and transmit a response from the patient.

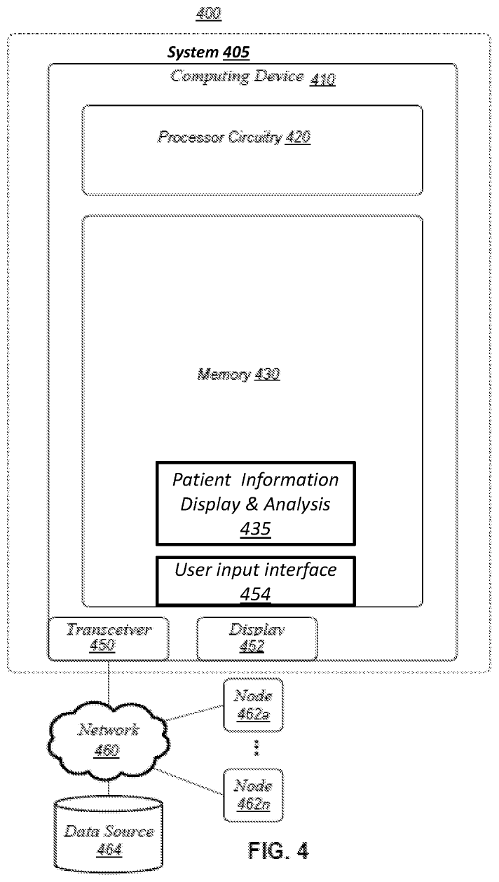

FIG. 4 illustrates an example of an operating environment 400 that may be representative of some implementations of the system described herein. As shown in FIG. 4, operating environment 400 may include a system 405 that is operative to perform network messaging functioning as described herein, and may include a system having network messaging functions that is further operative for treating patients, e.g., patients having chronic illnesses, such as a dialysis treatment or dialysis management device. In various embodiments, the system 405 may include a computing device 410. The computing device 410 may include processing circuitry 420, a memory unit 430, a transceiver 450, and/or a display 452. The processing circuitry 420 may be communicatively coupled to the memory unit 430, the transceiver 450, and/or the display 452. In some embodiments, the computing device 410 may be connected to a network 460 through transceiver 450. The network 460 may include nodes 462a-n, for example, remote computing devices, data sources 464, and/or the like.

The processing circuitry 420 may include and/or may access various logic for performing processes according to some embodiments. The processing circuitry 120, or portions thereof, may be implemented in hardware, software, or a combination thereof. As used in this application, the terms “logic, “component,” “layer,” “system,” “circuitry,” “decoder,” “encoder,” and/or “module” are intended to refer to a computer-related entity, either hardware, a combination of hardware and software, software, or software in execution. For example, a logic, circuitry, or a layer may be and/or may include, but are not limited to, a process running on a processor, a processor, a hard disk drive, multiple storage drives (of optical and/or magnetic storage medium), an object, an executable, a thread of execution, a program, a computer, hardware circuitry, integrated circuits, application specific integrated circuits (ASIC), programmable logic devices (PLD), digital signal processors (DSP), field programmable gate array (FPGA), a system-on-a-chip (SoC), memory units, logic gates, registers, semiconductor device, chips, microchips, chip sets, software components, programs, applications, firmware, software modules, computer code, combinations of any of the foregoing, and/or the like. It is also understood that components of the processing circuitry 420 may be located within an accelerator, a processor core, an interface, an individual processor die, implemented entirely as a software application and/or the like.

The memory unit 430 may include various types of computer-readable storage media and/or systems in the form of one or more higher speed memory units, such as read-only memory (ROM), random-access memory (RAM), dynamic RAM (DRAM), Double-Data-Rate DRAM (DDRAM), synchronous DRAM (SDRAM), static RAM (SRAM), programmable ROM (PROM), erasable programmable ROM (EPROM), electrically erasable programmable ROM (EEPROM), flash memory, polymer memory such as ferroelectric polymer memory, ovonic memory, phase change or ferroelectric memory, silicon-oxide-nitride-oxide-silicon (SONOS) memory, magnetic or optical cards, an array of devices such as Redundant Array of Independent Disks (RAID) drives, solid state memory devices (e.g., USB memory, solid state drives (SSD) and any other type of storage media suitable for storing information. In addition, memory unit 430 may include various types of computer-readable storage media in the form of one or more lower speed memory units, including an internal (or external) hard disk drive (HDD), a magnetic floppy disk drive (FDD), and an optical disk drive to read from or write to a removable optical disk (e.g., a CD-ROM or DVD), a solid state drive (SSD), and/or the like.

The memory unit 430 may store various information, e.g., one or more programs, to perform various functions identifying and treating patients with CKD and/or ESRD. In some embodiments, the memory unit 430 may include logic having application programming interfaces (APIs) and/or graphical user interfaces (GUIs) to read, write, and/or otherwise access information, such as via the display 452 and user input interface 454, web interfaces, mobile application (“mobile applications,” “mobile apps,” or “apps”), and/or the like.

In some embodiments, information stored in the memory unit 430 may be retrieved from and/or moved into a data source 464 including, without limitation, a hospital information management system (HIMS), laboratory information management system (LIMS), Health Information System (HIS), electronic medical records (EMR), a clinical trial database, and/or the like. For example, one or more programs or algorithms, or combinations thereof, as a patient information display and analysis 435, may be implementable. In some embodiments, treatment related information of the patient may be displayed via the information display and analysis 435 along with the emergency message of the system described herein, and for which patient responses may be process according to the indicated need of the patient in response to the emergency message. Specifically, according to the response the programs and/or algorithms may be utilized in connection with the safety and wellness functions of the network messaging system described herein. For example, an indicated patient response of needing assistance may be designated as urgent and thereby transmitted to a remote entity, via the system described herein, using an urgent or emergency transmission process, such as a special emergency communication channel or with appropriate characterization of the message (e.g. URGENT, or EMERGENCY) and routed to an appropriate receiving mailbox at the remote entity.

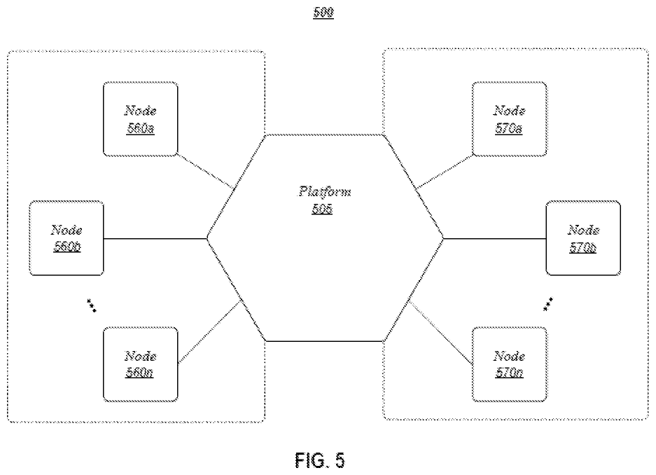

FIG. 5 illustrates an example of an operating environment 500 that may be representative of some implementations of the system described herein. The operating environment 500 may include a platform 505, e.g., a healthcare exchange platform. In some embodiments, the platform 505 may be operative to provide for the exchange of clinical data and/or clinical trial information among interested entities. In various embodiments, the platform 505 may include an application platform operative for identifying a patient population and treating CKD and/or ESRD with services among nodes 560a-n and 570a-n. In exemplary embodiments, the platform 505 may be a software platform, suite, set of protocols, and/or the like provided to customers by a manufacturer and/or developer (“developer”) associated with medical devices, medical care services, clinical research services, laboratory services, clinical trial services, and/or the like.

For example, a developer may provide the platform 505 as a data exchange interface for use by various entities, including government entities (for example, the FDA), and other stakeholders (for instance, pharmaceutical manufacturers, medical device manufacturers, and/or the like). An entity, such as a hospital, dialysis clinic, healthcare provider, government entity, regulatory entity, pharmaceutical manufacturer, medical device manufacturer, and/or the like providing and/or receiving clinical trial services via a node 570a-n provided by developer may use the platform 505 to implement processes according to some embodiments. Other entities may access the platform 505 via a GUI, such as a client application, web interface, mobile app, and/or the like, e.g., for performing functions associated with the memory 522. In some embodiments, at least a portion of the platform 505 may be hosted in a cloud computing environment.

Nodes 570a-n may be data producers for the memory 522 and nodes 560a-n may be data consumers of the memory 522. For example, node 570a-n may include entities providing clinical data, model information, and/or the like used by the memory 522 to generate, perform, and/or evaluate a patient population. Nodes 560a-n may include third-party applications, decision makers, analysis processes, regulators, and/or other data consumers that may be interested in the results of generating, performing, and/or evaluating the patient population. An entity may be both a data producer and a data consumer.

For example, node 560a may be care provider (node 560b) to provide treatment to a patient based on analysis of a patient population including medical records, laboratory data, pharmacy, and the like. (node 570a). Data producers 570a-n may provide analytical data, according to permissions, to the platform 505, for example, in the form of records in a HIMS, LIMS, EMR, and/or the like. Data consumers 560a-n may access analytical data, according to permissions, via the platform 505 (for example, through HIMS, LIMS, EMR, and/or the like and/or local copies of such records).

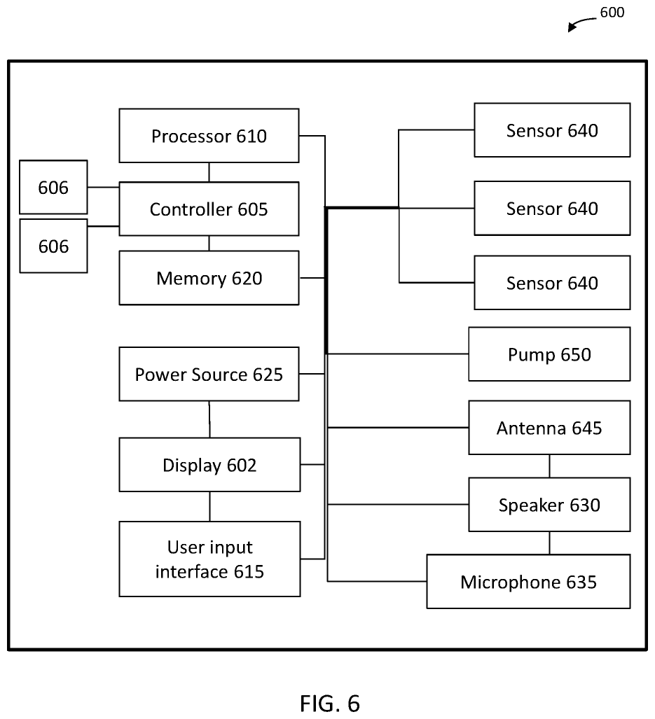

FIG. 6 is a schematic of an exemplary embodiment of a dialysis machine 600, and a controller 605 in accordance with the present disclosure are shown. The machine 600 may be a dialysis machine, e.g., a peritoneal dialysis machine or a hemodialysis machine, for performing a dialysis treatment on a patient. The controller 605 may automatically control execution of a treatment function during a course of dialysis treatment. The controller 605 may be operatively connected to sensors 640 and deliver one or more signals to execute one or more treatment functions, or a course of treatment associated with various treatment systems. Although FIG. 6 illustrates the components integrated into the dialysis machine 600, at least one of the controller 605, processor 610, and memory 620 may be configured to be external and wired or wirelessly connected to the dialysis machine 600, as an individual component of a dialysis system. In some embodiments the controller 605, processor 610 and memory 620 may be remote to the dialysis machine and configured to communicate wirelessly.

In some embodiments, the controller 605, processor 610, and memory 620 of the system or machine 600, may receive signals from sensor 640 indicating one or more patient parameters. Communication between the controller 605 and the treatment system may be bi-directional, whereby the treatment system acknowledges control signals, and/or may provide state information associated with the treatment system and/or requested operations. For example, system state information may include a state associated with specific operations to be executed by the treatment system (e.g., trigger pump to deliver dialysate, trigger pumps and/or compressors to deliver filtered blood, and the like) and a status associated with specific operations (e.g., ready to execute, executing, completed, successfully completed, queued for execution, waiting for control signal, and the like).

The dialysis system or machine 600 may also include at least one pump 650 operatively connected to the controller 605. The controller 605 may also be operatively connected to one or more speakers 630 and one or more microphones 635 disposed in the system or machine 600. The user input interface 615 may include a combination of hardware and software components that allow the controller 605 to communicate with an external entity, such as a patient or other user. These components may be configured to receive information from actions such as physical movement or gestures and verbal intonation. In embodiments, the components of the user input interface 615 may provide information to external entities. Examples of the components that may be employed within the user input interface 615 include keypads, buttons, microphones, touch screens, gesture recognition devices, display screens, and speakers.

As shown in FIG. 6, sensors 640 may be included for detecting and monitoring one or more parameters and be operatively connected to at least the controller 605, processor 610, and memory 620. The processor 610 may be configured to execute an operating system, which may provide platform services to application software, e.g., for operating the dialysis machine 600. These platform services may include inter-process and network communication, file system management and standard database manipulation. One or more of many operating systems may be used, and examples are not limited to any particular operating system or operating system characteristic. In some examples, the processor 610 may be configured to execute a real-time operating system (RTOS), such as RTLinux, or a non-real time operating system, such as BSD or GNU/Linux. According to a variety of examples, the processor 610 may be a commercially available processor such as a processor manufactured by INTEL, AMD, MOTOROLA, and FREESCALE. However, the processor 610 may be any type of processor, multiprocessor or controller, whether commercially available or specially manufactured. For instance, according to one example, the processor 610 may include an MPC823 microprocessor manufactured by MOTOROLA.

The memory 620 may include a computer readable and writeable nonvolatile data storage medium configured to store non-transitory instructions and data. In addition, the memory 620 may include a processor memory that stores data during operation of the processor 610. In some examples, the processor memory includes a relatively high performance, volatile, random access memory such as dynamic random access memory (DRAM), static memory (SRAM), or synchronous DRAM. However, the processor memory may include any device for storing data, such as a non-volatile memory, with sufficient throughput and storage capacity to support the functions described herein. Further, examples are not limited to a particular memory, memory system, or data storage system.

The instructions stored on the memory 620 may include executable programs or other code that may be executed by the processor 610. The instructions may be persistently stored as encoded signals, and the instructions may cause the processor 610 to perform the functions described herein. The memory 620 may include information that is recorded, on or in, the medium, and this information may be processed by the processor 610 during execution of instructions. The memory 620 may also include, for example, specification of data records for user timing requirements, timing for treatment and/or operations, historic sensor information, and other databases and the like. The medium may, for example, be optical disk, magnetic disk or flash memory, among others, and may be permanently affixed to, or removable from, the controller 600.

The controller 605 may be disposed in the machine 600 or may be coupled to the machine 600 via a communication port or wireless communication links, shown schematically as communication element 606. For example, the communication element 606 may connect the dialysis machine 600 to the network messaging system 300 described herein or another remote system such as an outside system or other clinical system. The dialysis machine 600 may be connectable to the network messaging system 300 via the communication element 606 so that the controller 605 may send and receive information and other signals in accordance with the safety and wellness confirmations described herein.

As used in this application, the terms “system” and “component” and “module” are intended to refer to a computer-related entity, either hardware, a combination of hardware and software, software, or software in execution, examples of which are provided by the exemplary computing architecture. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers. Further, components may be communicatively coupled to each other by various types of communications media to coordinate operations. The coordination may involve the uni-directional or bi-directional exchange of information. For instance, the components may communicate information in the form of signals communicated over the communications media. The information can be implemented as signals allocated to various signal lines. In such allocations, each message is a signal. Further embodiments, however, may alternatively employ data messages. Such data messages may be sent across various connections. Exemplary connections include parallel interfaces, serial interfaces, and bus interfaces.

Implementations discussed herein may be combined with each other in appropriate combinations in connection with the system described herein. Additionally, in some instances, the order of steps in the flow diagrams, flowcharts and/or described flow processing may be modified, where appropriate. The system may further include a display and/or other computer components for providing a suitable interface with a user and/or with other computers. Aspects of the system described herein may be implemented or controlled using software, hardware, a combination of software and hardware and/or other computer-implemented or computer-controlled modules or devices having described features and performing described functions. Data exchange and/or signal transmissions to, from and between components of the system may be performed using wired or wireless communication. This communication may include use of one or more transmitter or receiver components that securely exchange information via a network, such as the Internet, and may include use of components of local area networks (LANs) or other smaller scale networks, such as Wi-Fi, Bluetooth or other short range transmission protocols, and/or components of wide area networks (WANs) or other larger scale networks, such as mobile telecommunication networks.

Software implementations of aspects of the system described herein may include executable code that is stored in a computer-readable medium and executed by one or more processors. The computer-readable medium may include volatile memory and/or non-volatile memory, and may include, for example, a computer hard drive, ROM, RAM, flash memory, portable computer storage media, an SD card, a flash drive or other drive with, for example, a universal serial bus (USB) interface, and/or any other appropriate tangible or non-transitory computer-readable medium or computer memory on which executable code may be stored and executed by a processor. The system described herein may be used in connection with any appropriate operating system. The meanings of any method steps of the invention(s) described herein are intended to include any suitable method of causing one or more parties or entities to perform the steps unless a different meaning is expressly provided or otherwise clear from the context.

Some embodiments of the disclosed systems may be implemented, for example, using a storage medium, a computer-readable medium or an article of manufacture which may store an instruction or a set of instructions that, if executed by a machine (i.e., processor or microcontroller), may cause the machine to perform a method and/or operations in accordance with embodiments of the disclosure. In addition, a server or database server may include machine readable media configured to store machine executable program instructions. Such a machine may include, for example, any suitable processing platform, computing platform, computing device, processing device, computing system, processing system, computer, processor, or the like, and may be implemented using any suitable combination of hardware, software, firmware, or a combination thereof and utilized in systems, subsystems, components, or sub-components thereof. The computer-readable medium or article may include, for example, any suitable type of memory unit, memory device, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, and the like, implemented using any suitable high-level, low-level, object-oriented, visual, compiled and/or interpreted programming language.

Numerous specific details have been set forth herein to provide a thorough understanding of the embodiments. It will be understood by those skilled in the art, however, that the embodiments may be practiced without these specific details. In other instances, well-known operations, components, and circuits have not been described in detail so as not to obscure the embodiments. It can be appreciated that the specific structural and functional details disclosed herein may be representative and do not necessarily limit the scope of the embodiments. Some embodiments may be described using the expression “coupled” and “connected” along with their derivatives. These terms are not intended as synonyms for each other. For example, some embodiments may be described using the terms “connected” and/or “coupled” to indicate that two or more elements are in direct physical or electrical contact with each other. The term “coupled,” however, may also mean that two or more elements are not in direct contact with each other, but yet still co-operate or interact with each other.

Unless specifically stated otherwise, it may be appreciated that terms such as “processing,” “computing,” “calculating,” “determining,” or the like, refer to the action and/or processes of a computer or computing system, or similar electronic computing device, that manipulates and/or transforms data represented as physical quantities (e.g., electronic) within the computing system's registers and/or memories into other data similarly represented as physical quantities within the computing system's memories, registers or other such information storage, transmission or display devices. The embodiments are not limited in this context.

It should be noted that the methods described herein do not have to be executed in the order described, or in any particular order. Moreover, various activities described with respect to the methods identified herein can be executed in serial or parallel fashion.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. It is to be understood that the above description has been made in an illustrative fashion, and not a restrictive one. Combinations of the above embodiments, and other embodiments not specifically described herein will be apparent to those of skill in the art upon reviewing the above description. Thus, the scope of various embodiments includes any other applications in which the above compositions, structures, and methods are used.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. As used herein, an element or operation recited in the singular and proceeded with the word “a” or “an” should be understood as not excluding plural elements or operations, unless such exclusion is explicitly recited. Furthermore, references to “one embodiment” of the present disclosure are not intended to be interpreted as excluding the existence of additional embodiments that also incorporate the recited features. To the extent used in this description and in the claims, a recitation in the general form of “at least one of [a] and [b]” should be construed as disjunctive. For example, a recitation of “at least one of [a], [b], and [c]” would include [a] alone, [b] alone, [c] alone, or any combination of [a], [b], and [c].

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Furthermore, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes.

1. A notification system, comprising:
a connected health system comprising a network system that enables secure transmission of information between a patient device and a remote entity; and
a notification tool comprising an application having a broadcaster access interface and a recipient access interface, wherein the notification tool is configured to allow a user at the remote entity to create an emergency message, send the emergency message via the connected health system to multiple recipients at the same time, and manage an interface between a messaging application and the messaging access platform, wherein the emergency message requests a response from the recipient that is entered by the recipient via the recipient access interface.

2. The notification system according to claim 1, wherein the emergency message is a patient safety check request or a patient well-being confirmation request.

3. The notification system according to claim 1, wherein the notification tool includes a web-based application.

4. The notification system according to claim 1, wherein the notification tool further comprises an interface to a workflow application.

5. The notification system according to claim 1, wherein the patient device is a mobile device, and wherein the recipient access interface is installed on the mobile device.

6. The notification system according to claim 5, wherein the mobile device is a smart phone or tablet device.

7. The notification system according to claim 1, wherein the patient device is a treatment device having a display, and wherein the recipient access interface is installed on the treatment device.

8. The notification system according to claim 7, wherein the treatment device is a dialysis machine.

9. The notification system according to claim 8, wherein the display of the dialysis machine displays information related to a treatment performed by the dialysis machine and the emergency message.

10. The notification system according to claim 1, wherein the notification tool further comprises a response processing component that processes responses received in response to the emergency message and facilitates responsive action.

11. A medical system, comprising:
a medical device that is configured to perform a medical treatment;
a connected health system comprising a network system that enables secure transmission of information between the medical device and a remote entity, wherein the information includes health related information; and
a notification tool comprising an application having a broadcaster access interface and a recipient access interface, wherein the notification tool is configured to allow a user at the remote entity to create an emergency message, send the emergency message via the connected health system to multiple recipients at the same time, and manage an interface between a messaging application and the messaging access platform, wherein the emergency message requests a response from the recipient that is entered by the recipient via the recipient access interface.

12. The medical system according to claim 11, wherein the emergency message is a patient safety check request or a patient well-being confirmation request.

13. The medical system according to claim 11, wherein the notification tool includes a web-based application.

14. The medical system according to claim 11, wherein the notification tool further comprises an interface to a workflow application.

15. The medical system according to claim 11, further comprising a mobile device coupled to the medical device, wherein the recipient access interface is installed on the mobile device.

16. The medical system according to claim 15, wherein the mobile device is a smart phone or tablet device.

17. The medical system according to claim 11, wherein the medical device is a treatment device having a display, and wherein the recipient access interface is installed on the medical device.

18. The medical system according to claim 17, wherein the treatment device is a dialysis machine.

19. The medical system according to claim 18, wherein the display of the dialysis machine displays information related to a treatment performed by the dialysis machine and the emergency message.

20. The medical system according to claim 11, wherein the notification tool further comprises a response processing component that processes responses received in response to the emergency message and facilitates responsive action.
